(19) United States
(12) Patent Application Publication
(10) Pub. No.: US 2003/0074030 A1
(43) Pub. Date: Apr. 17, 2003

(54) Method and apparatus for controlling percutaneous electrical signals

(75) Inventors: Kent Leyde, Sammamish, WA (US); Jon M. Bishay, Woodinville, WA (US); John F. Harris, Bellevue, WA (US)

Correspondence Address: PERKINS COIE LLP, PATENT-SEA, P.O. BOX 1247, SEATTLE, WA, 98111-1247, US

(73) Assignee: Vertis Neuroscience, Inc.

(21) Appl. No.: 10/260,058

(22) Filed: Sep. 27, 2002

Related U.S. Application Data
(60) Provisional application No. 60/325,725, filed on Sep. 28, 2001.

(51) Int. Cl. A61N 1/18

(52) U.S. Cl. 607/27

(57) ABSTRACT

Methods and apparatuses for controlling percutaneous electrical signals. In one embodiment, the method can include a computer-implemented method for displaying characteristics of an electrical therapy signal, which can include receiving a first value for a characteristic of an electrical therapy signal and directing transmission of a first electrical therapy signal with the first value. The method can further include directing a digital display device to display a first graph representing the first value, receiving a second value for the characteristic, directing transmission of a second electrical therapy signal with the second value, and directing the digital display device to display a second graph concurrent with a display of the first graph. The apparatus can simultaneously control signal levels directed to multiple channels, and can automatically set the signal levels to zero when a therapy session is ended or paused.

### CROSS-REFERENCE TO RELATED APPLICATION

The present application claims priority to U.S. Provisional Patent Application 60/325,725 (attorney docket 33734.8016US00) filed Sep. 28, 2001 and incorporated herein in its entirety by reference.

### BACKGROUND

The present invention is directed to methods and apparatuses for controlling electrical signals delivered percutaneously to a recipient. Electrical therapy has been used conventionally in medicine to treat pain and other conditions. For example, transcutaneous electrical nerve stimulation (TENS) systems deliver electrical therapy through electrode patches placed on the surface of a patient's skin to treat pain in tissue beneath and around the location of the patches. One drawback with TENS systems is that they may not provide patients with adequate relief. One approach to addressing this drawback has been to deliver therapeutic electrical current percutaneously using a percutaneous neuromodulation therapy (PNT) technique. This technique can include inserting a sharpened electrode through the patient's skin to a location near a target nerve, then coupling the electrode to a source of modulated electrical current.

One feature of conventional PNT techniques is that they typically require that the practitioner simultaneously deliver electrical current through a number of channels to a corresponding number of sites on the patient's body. One drawback with this technique is that it can be difficult for the practitioner to easily monitor the electrical current levels applied to each channel. Another drawback with this feature is that it may be time consuming for the practitioner to manually adjust the current levels for each channel. Still another drawback is that it may be difficult for the practitioner to pause the therapy session (for example, to give the patient a break) and then restart the therapy session at current levels approximating those administered to the patient when the session was paused.

### SUMMARY

The present invention is directed to methods and apparatuses for controlling electrical signals delivered percutaneously to a recipient. A computer-implemented method in accordance with one aspect of the invention can include receiving a first value for a characteristic of an electrical therapy signal and directing transmission of a first electrical therapy signal with the first value of the characteristic. The method can further include directing a digital display device to display a first graph representing the first value of the characteristic. The method can further include receiving a second value for the characteristic, directing transmission of a second electrical therapy signal with the second value, and directing the digital display device to display a second graph concurrent with a display of the first graph and representing the second value of the characteristic. In a further aspect of this embodiment, the second graph can have a color and/or intensity different than a corresponding color and/or intensity of the first graph.

The invention is also directed toward an apparatus for controlling therapeutic electrical signals transmitted to a recipient. The apparatus can include a therapeutic signal emitter operatively coupled to a signal controller. The signal controller can include a first input device coupled to a first channel to control a signal level directed to the first channel, and can further include a second input device coupled to a second channel to control a signal level directed to the second channel independently of the signal directed to the first channel. The signal controller can still further include a third or common input device coupled to both the first and second channels to simultaneously control a single signal level directed to both the first and second channels.

In another aspect of the invention, a signal input device can be operatively coupled to a signal emitter to control a current of the electrical signal emitted by the signal emitter. The signal emitter can be changed from an unpowered state to a powered state. The signal input device can be movable between a first position and a second position, with the first position corresponding to a first non-zero electrical signal current, and the second position corresponding to a second non-zero electrical signal current. In one embodiment, the signal emitter emits no electrical signal upon being placed in the powered state, whether the signal input device is in the first position or the second position, until the signal input device is manipulated.

### BRIEF DESCRIPTION OF THE DRAWINGS

FIG. 1 is a partially schematic isometric view of an apparatus positioned proximate to a recipient in accordance with an embodiment of the invention.

FIG. 2 is an isometric view of a control device configured to control electrical signals directed to the recipient in accordance with an embodiment of the invention.

FIG. 3 is a rear view of an embodiment of the control unit shown in FIG. 2.

FIGS. 4-5D illustrate graphical displays on a display screen of the control device shown in FIGS. 1-3 during self-test and set up in accordance with further embodiments of the invention.

FIGS. 6-14A illustrate graphical displays on a display screen of the control device shown in FIGS. 1-3 during operation in accordance with still further embodiments of the invention.

FIG. 14B is a flowchart illustrating a method for presenting a graphical display such as that shown in FIG. 14A in accordance with an embodiment of the invention.

FIG. 15 is a graphical display in accordance with another embodiment of the invention.

### DETAILED DESCRIPTION

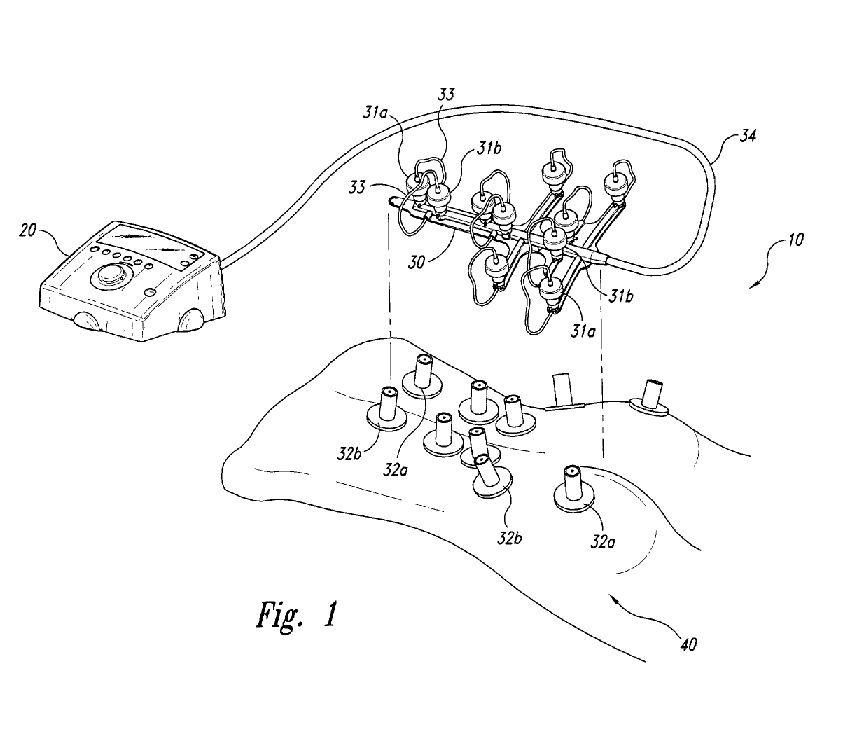

The present disclosure describes methods and apparatuses for controlling electrical signals delivered percutaneously to a recipient. Many specific details of certain embodiments of the invention are set forth in the following description and in FIGS. 1-15 to provide a thorough understanding of these embodiments. One skilled in the art, however, will understand that the present invention may have additional embodiments, and that the invention may be practiced without several of the details described below.

FIG. 1 is a partially schematic, isometric illustration of an apparatus 10 configured to deliver percutaneous electrical therapy to a recipient 40. In one aspect of this embodiment, the apparatus 10 can include a plurality of probe assemblies 32 (shown as first probe assemblies 32a and second probe assemblies 32b) removably attached to the recipient 40. Each probe assembly 32 can include a percutaneous probe (not visible in FIG. 1) that can be removably inserted into the recipient 40 during a therapy session. In one embodiment, the probe can include a percutaneous electrode and in other embodiments, the probe can include other percutaneous devices.

The apparatus 10 can further include a corresponding plurality of probe couplers 31 (shown in FIG. 1 as first probe couplers 31a and second probe couplers 31b), each configured to be coupled to a corresponding one of the probe assemblies 32. Each probe coupler 31 can be removably supported on a support member 30 which can be placed on the recipient's back during a therapy session. The practitioner can remove each probe coupler 31 and engage it with a corresponding probe assembly 32 to (a) insert the percutaneous probe of the probe assembly 32 into the recipient, and, when the percutaneous probes include percutaneous electrodes, (b) provide an electrical connection between the probe and a coupler cable 33 that extends between the coupler 31 and the support member 30.

A support member cable 34 can extend between the support member 30 and the control device 20 to transmit electrical signals from the control device 20 to the probes via the coupler cables 33 and the probe couplers 31. Accordingly, each pair of probe assemblies 32a, 32b can provide a supply and return path for therapeutic electrical current delivered to the recipient 40. In a further aspect of this embodiment, each pair of probe assemblies 32a, 32b can be coupled to an individually controllable channel of the control device 20, as described in greater detail below. Accordingly, the control device 20 can provide treatment that accommodates differences in sensitivities among treatment sites on the recipient's body, and among different recipients. Further details of aspects of the probe assembly 32, the probe coupler 31, and the support member 30 are included in the following pending U.S. patent applications: 09/457,477, titled “Percutaneous Electrical Therapy System with Electrode Entry Angle Control,” filed Dec. 1, 1999; 09/666,931, titled “Method and Apparatus for Repositioning a Percutaneous Probe,” filed Sep. 21, 2000; 09/928,044, titled “Method and Apparatus for Deploying a Percutaneous Probe,” filed Aug. 11, 2001; and 09/751,382, titled “Apparatus and Method for Coupling Therapeutic and/or Monitoring Equipment to a Patient,” filed Dec. 29, 2000. The foregoing patent applications are incorporated herein in their entireties by reference.

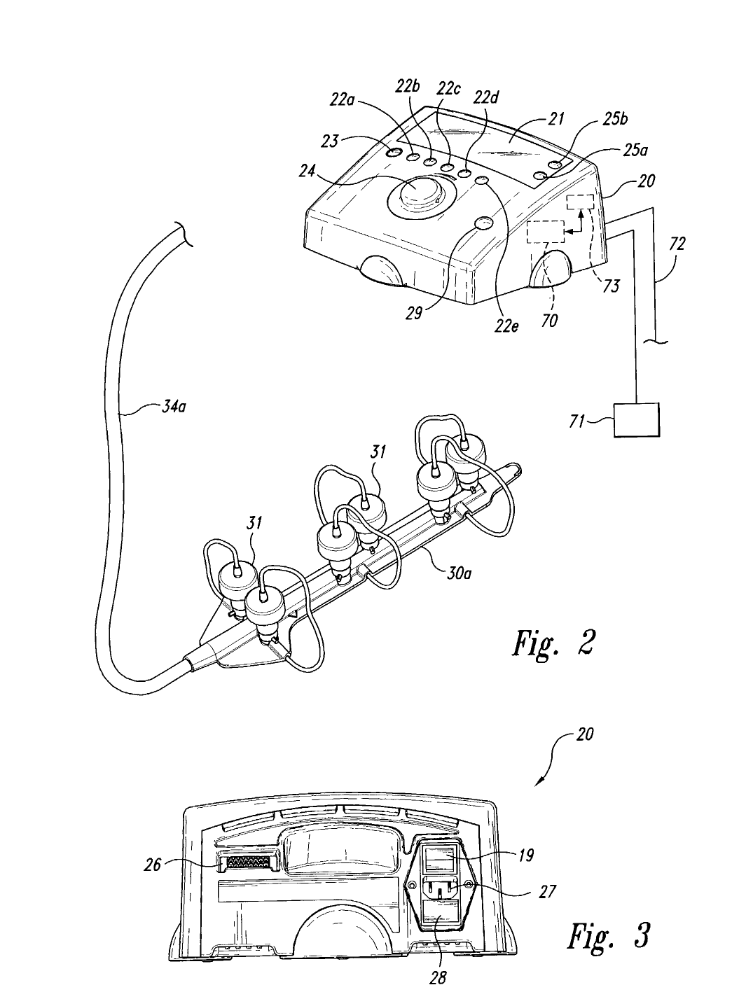

In one embodiment, the control device 20 can be configured to connect (via the support member cable 34) to a support member 30 that supports five pairs of probe couplers 31 on the recipient's back. In other embodiments, the same control device 20 can connect to other support members that deliver therapy to other parts of the body and/or rest on other parts of the body, via the same or a different support member cable 34. For example, as shown in FIG. 2, the control device 20 can connect (via a support member cable 34a) to a support member 30a having three pairs of probe couplers 31. Accordingly, the control device 20 can be compatible with a variety of support members and associated cables and can be suitable for delivering therapy to a variety of locations on the recipient's body.

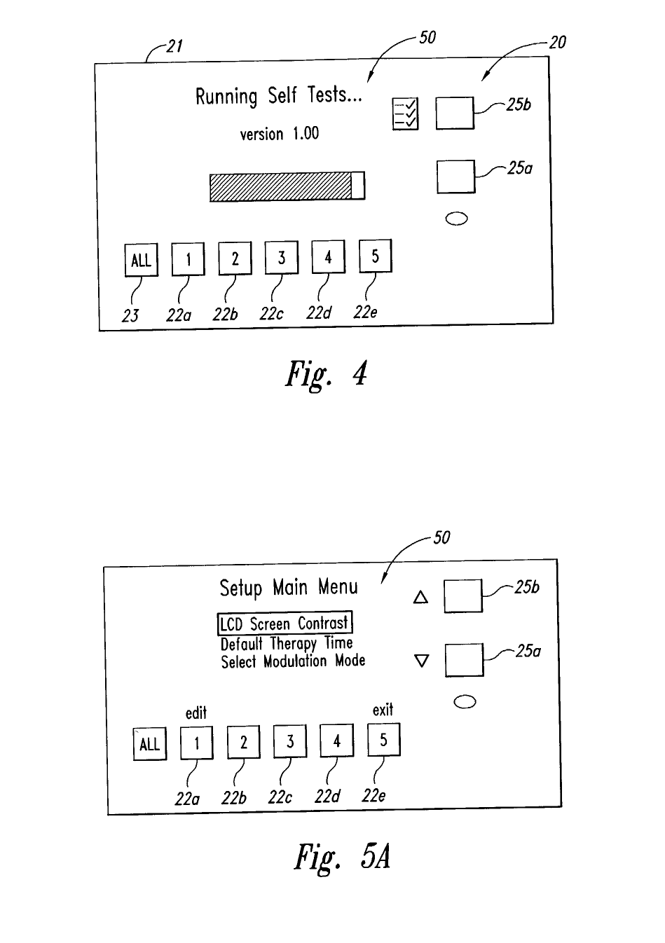

As is also shown in FIG. 2, the control device 20 can include a processor 70 that automatically (often with input from the practitioner) performs many or all of the functions described below with reference to FIGS. 4-15. The control device 20 can also include a display screen 21 configured to present a variety of graphical displays to the practitioner during the course of a therapy session. In one embodiment, the display screen 21 can include an LCD device. In other embodiments, the display screen 21 can include other devices, such as a CRT display, and LED display, and/or an electro-luminescent display.

In other embodiments, the control device 20 can include other output devices in addition to or in lieu of the display screen 21. For example, the control device can include an internal or external printer 71 (shown schematically in FIG. 2) to print results and/or diagnostic information associated with a therapy session. Such information can also be stored internally in a memory 73 of the control device 20 and/or transmitted to other locations and/or devices via a communication link 72. The communication link 72 can also be used to perform service, diagnostics, and/or maintenance on the control device 20 from a remote location.

The control device 20 can further include a plurality of input devices, such as channel select buttons 22 (five are shown in FIG. 2 as channel select buttons 22a-e), each configured to selectively control characteristics of electrical signals applied to the pairs of probe assemblies 32a, 32b described above with reference to FIG. 1. An all-channel button 23 can be operated to apply the same input command to all channels simultaneously. The input command can be made by rotating a control knob 24, for example, to adjust the level of electrical current applied to each channel. Other input commands can be made by depressing function or input buttons 25 (shown as first and second function buttons 25a, 25b) and/or a pause button 29, as described in greater detail below with reference to FIGS. 4-15.

In one aspect of this embodiment, the control device 20 can automatically sense which type of support member and support member cable it is connected to, and activate or de-activate the corresponding select buttons 22 accordingly. For example, when the control device 20 is coupled to the support member 30 shown in FIG. 1, all five channel select buttons 22a-e can be active. When the control device 20 is coupled to the support member 30a shown in FIG. 2, three of the five channel select buttons (for example, buttons 22a-c) can be active.

FIG. 3 is a rear elevation view of an embodiment of the control device 20. The control device 20 can include a cable connector 26 for coupling to the support member cable 34 (FIG. 1) or 34a (FIG. 2). The control device 20 can further include a power socket 27 for receiving power, an on/off switch 19, and a fuse box 28 to provide overload protection to the control device 20 in a conventional manner.

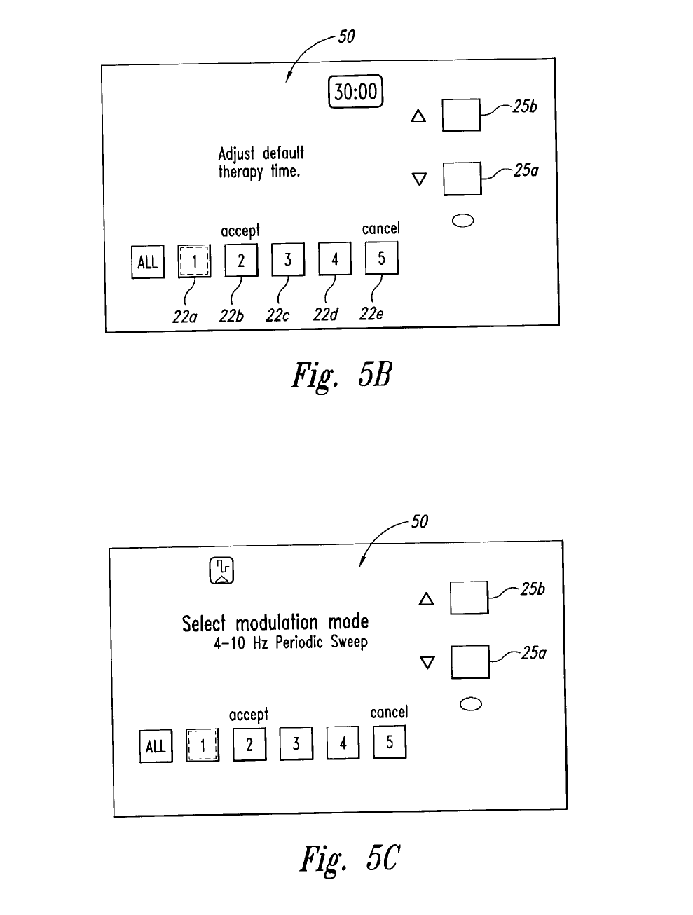

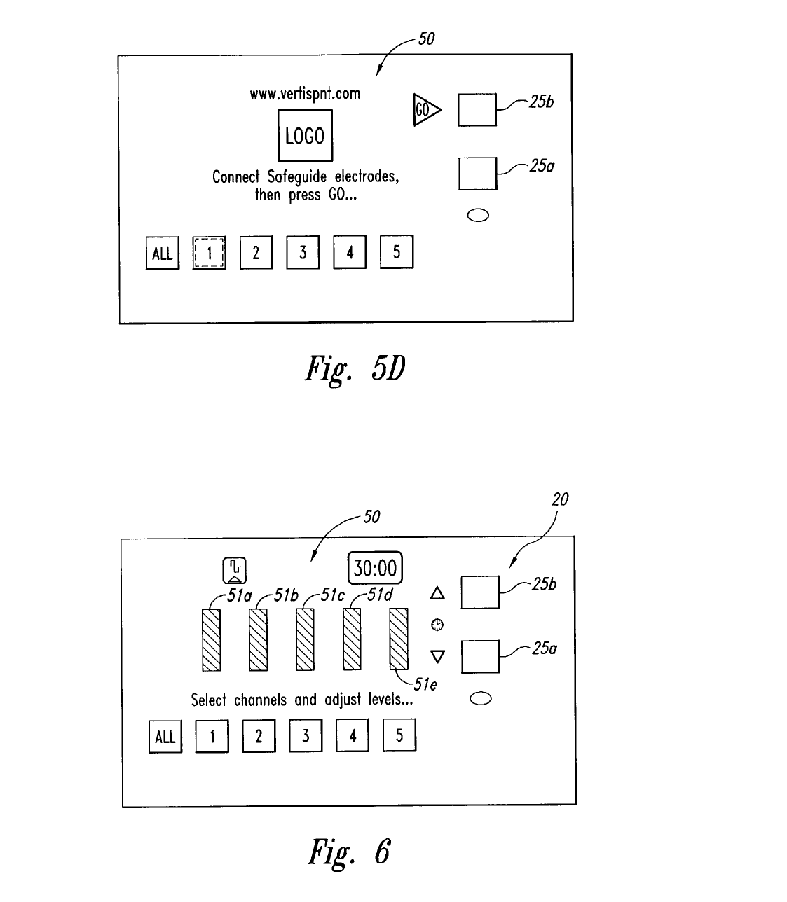

FIG. 4 is a partially schematic illustration of a portion of the control device 20, including the display screen 21. Also shown in FIG. 4 are the all-channel button 23, the channel select buttons 22a-e, and the function buttons 25a, 25b. These buttons can be square or rectangular as shown in FIG. 4, or circular as shown in FIGS. 1 and 2, or these buttons can have other shapes in other embodiments. In any of these embodiments, the display screen 21 can show a graphical display 50 that provides the practitioner with information regarding the status of the therapy session. As shown in FIG. 4, the graphical display 50 can indicate that the control device 20 is running self-tests, for example, when the control device 20 is initially changed from an unpowered state to a powered state. If the practitioner presses the second function button 25b during the self-test, the practitioner can set up default parameters for subsequent therapy sessions, as described below with reference to FIGS. 5A-D.

As shown in FIG. 5A, the graphical display 50 can next display a setup menu through which the practitioner can scroll by pressing the first and second function buttons 25a, 25b. The practitioner can edit or act on a particular item on the menu by pressing the first channel select button 22a, and can exit the menu by pressing the fifth channel select button 22e. In one embodiment, the practitioner can choose from adjusting the contrast for the display screen 21, adjusting the default therapy time for each session, and/or selecting a modulation mode for the current applied to the recipient, as described in greater detail below with reference to FIGS. 5B-C.

FIG. 5B illustrates the graphical display 50 as it can appear when the practitioner has selected to edit the default therapy time menu item shown in FIG. 5A. The practitioner can adjust the default therapy time up or down using the function buttons 25a, 25b, and can accept the updated default therapy time by pressing the second channel select button 22b, or cancel the selection by pressing the fifth channel select button 22e.

As shown in FIG. 5C, the graphical display 50 can display a variety of modulation modes (one is shown in FIG. 5C as a 4-10 Hz periodic sweep mode) from which the practitioner can select. The practitioner can scroll up and/or down through the list of modes by pressing the function buttons 25a, 25b. and can accept a selection by pressing the second channel select button 22b, or cancel the selection by pressing the fifth channel select button 22e. Additional modulation modes can include a continuous current level at 4 Hz or 50 Hz, a current level that alternates between 15 Hz and 30 Hz, or a current level that sweeps in an aperiodic manner between 4 Hz and 50 Hz. Further details of additional modulation nodes are included in the following pending U.S. patent applications: 09/686,993, titled “System and Method for Providing Percutaneous Electrical Therapy,” filed Oct. 10, 2000; and 09/751,503, titled “System and Method for Varying Characteristics of Electrical Therapy,” filed Dec. 29, 2000; both incorporated herein in their entireties by reference.

In an alternate embodiment, the control device 20 can automatically select the mode and the maximum output current level (as well as the number of output channels) based on the type of support member 30 of FIG. 1) and support member cable 34 (FIG. 2) connected to the control device 20. Accordingly, the control device 20 can recognize the type of support member and/or cable connected to it based, for example, on a signal received from the cable.

In one embodiment, the support member cable 34 can include an identity device 80. In one aspect of this embodiment, the identity device 80 can include an integrated circuit chip that stores a pre-selected, unique identifier, such as a serial number. For example, the serial number can be the same for all support member cables 34 that are pre-determined to be compatible with a corresponding control device 20. Suitable identity devices 80 are manufactured by Maxim Integrated Products of Sunnyvale, Calif. and Dallas Semiconductor of Dallas, Tex. In other embodiments, the identity device 80 can have other configurations. Further details of aspects of these devices are included in U.S. Provisional Application 60/325,975 (attorney docket 33734.8015US00) filed Sep. 28, 2001 and incorporated herein in its entirety by reference.

In any of the foregoing embodiments, the identifier provided by the identity device 80 can be correlated with the type of support member 30. For example, one identifier can be correlated with a clavicular support member, and another identifier can be correlated with a lumbar support member. In other embodiments, the identifier can be correlated with other types of support members. In any of these embodiments, the control device 20 can automatically select (a) the number of channels to which therapeutic signals are directed, (b) the mode and/or manner in which the therapeutic signals are applied, and/or (c) the maximum current level applied to the channels, based on information provided by the identity device 80.

When the self-test has been completed, the graphical display 50 can instruct the practitioner (as shown in FIG. 5D) to connect the probe couplers 31 (FIG. 1) to the corresponding probe assemblies 32 (FIG. 1). The practitioner can then initiate a therapy session by pressing the second function button 25b.

FIG. 6 illustrates the graphical display 50 after the practitioner has initiated the therapy session. The graphical display 50 can include a plurality of output bars 51 (shown as output bars 51a-e), each of which corresponds to one of the electrical signal channels. Alternatively, the graphical display 50 can include another graphical and/or pictorial representation to depict a characteristic of a signal applied to the electrical signal channels. In any of these embodiments, the representation (such as the output bars 51) can initially appear in a muted or gray-scale tone to indicate that an electrical current level has not yet been selected for any of the corresponding output channels. In other embodiments, the output bars 51 can have other initial characteristics to indicate that the current level has not yet been established. In a further aspect of this embodiment, the current level applied to each channel can automatically be set to zero when the control device 20 is powered up at the outset of a therapy session, regardless of the position of the control knob 24 (FIG. 2).

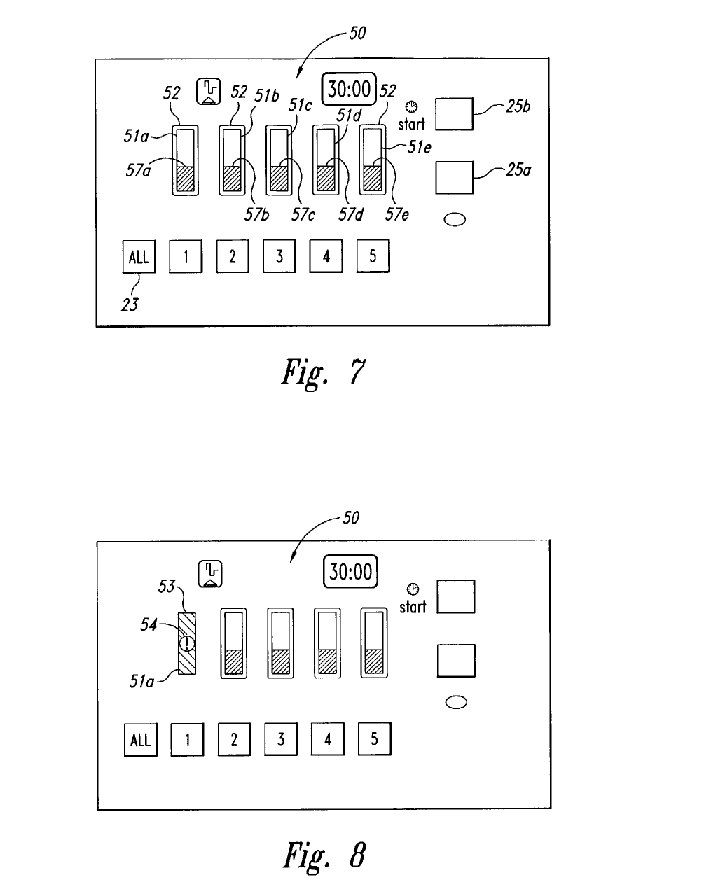

As shown in FIG. 7, the practitioner can simultaneously adjust the current level for each of the channels by pressing the all-channel button 23 and rotating the control knob 24 (FIG. 2). Each output bar 51 is then surrounded by an active frame 52. As the practitioner adjusts the control knob 24, five present current bars 57 (shown as bars 57a-e) expand and contract in a vertical direction to indicate the present level of current applied to each corresponding channel.

FIG. 8 illustrates the graphical display 50 as it can appear when a fault has been detected with one of the channels. As shown in FIG. 8, the output bar 51a corresponding to the faulty channel has an inactive frame 53 surrounding it, and the active frame 52 (FIG. 7) no longer appears. An alert icon 54 can appear in the middle of the faulty output bar 51a, and the color of the faulty output bar 51a can be muted or reduced in intensity relative to the present current bars 57. In other embodiments, the faulty channel can be highlighted in other manners.

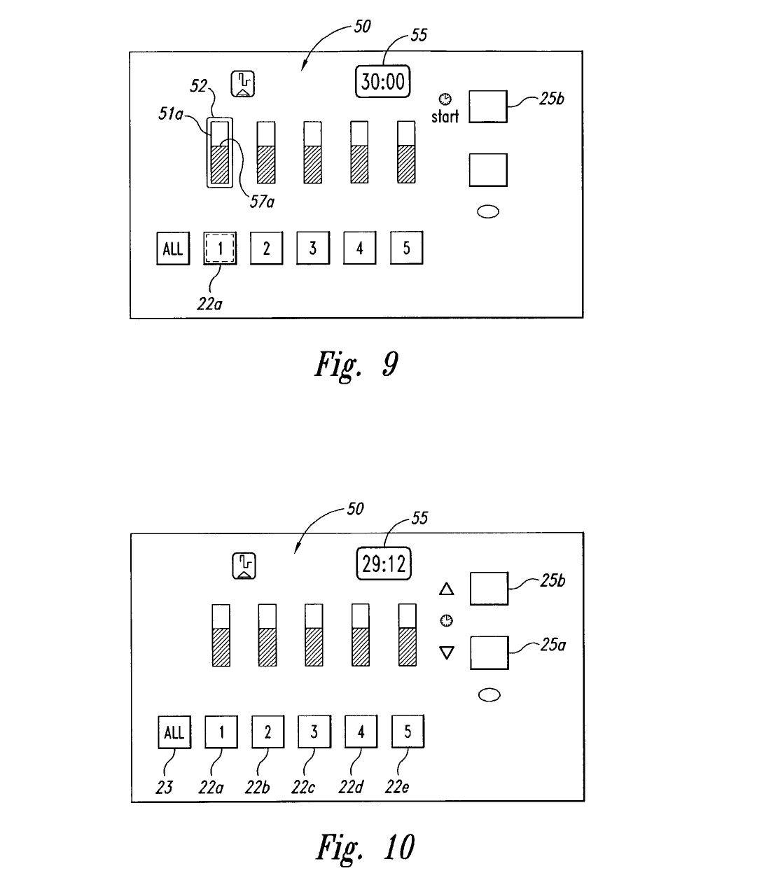

FIG. 9 illustrates the graphical display 50 as it appears when the practitioner selects an individual one of the channels for manipulation. For example, if the practitioner selects the first channel select button 22a, only the first output bar 51a has an active frame 52 surrounding it, and only the present current bar 57a increases and decreases in size as the practitioner rotates the control knob 24 (FIG. 2). Accordingly, the practitioner can individually adjust the current level for any of the output channels without affecting the current level for the remaining channels. Once the current levels have been selected for all channels, the practitioner can begin delivery of electrical current to the recipient by pressing the second function button 25b. A timer window 55 displays the time at the outset of the therapy session.

Referring now to FIG. 10, the timer window 55 can display the time remaining in the therapy session after the therapy session has been initiated. The practitioner can manually decrease or increase the amount of time remaining in the session by pressing the first function button 25a or the second function button 25b, respectively. As the therapy session progresses, the practitioner can also manually adjust the current level applied to the recipient in a manner generally similar to that described above, by operating the control knob 24 (FIG. 2) in conjunction with either the all-channel button 23 or one or more of the channel select buttons 22a-e.

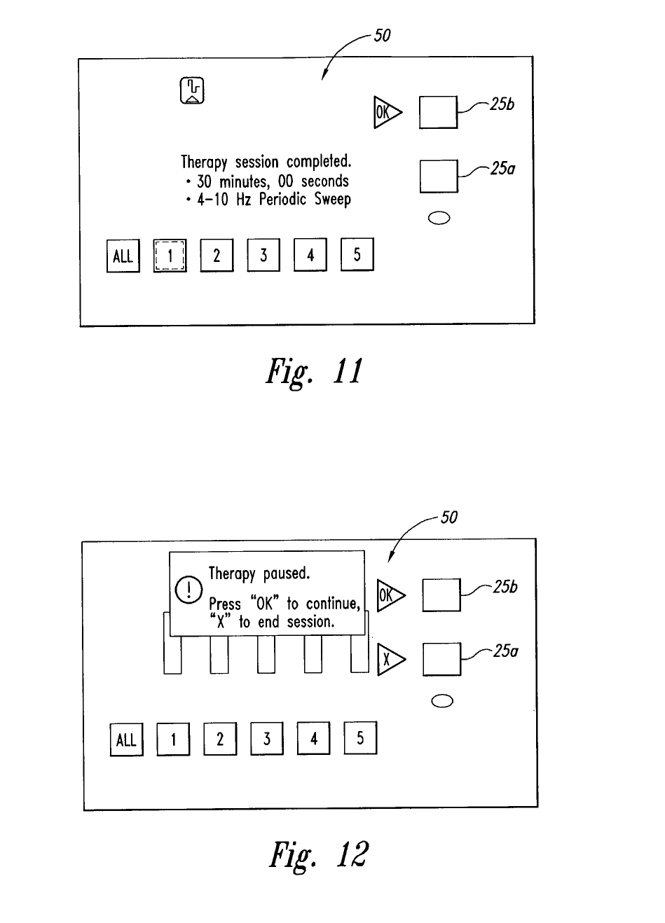

At the end of the session (referring now to FIG. 11), the graphical display 50 can display a text message indicating that the therapy session has been completed. The message can further indicate how long the therapy session was and the manner in which the current applied to the recipient was modulated.

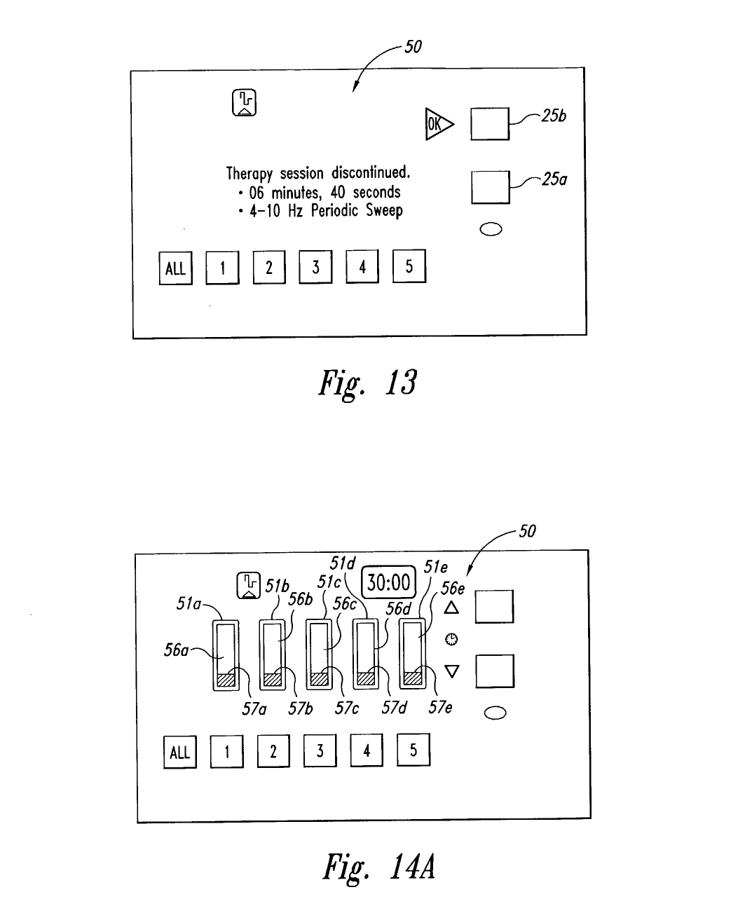

Referring now to FIG. 12, the practitioner can interrupt the therapy session prior to its normal termination if desired. For example, the practitioner can press the pause button 29 (FIG. 2) to interrupt the session. The graphical display 50 can then display a message indicating that the therapy session has been paused. If the practitioner wishes to end the therapy session, he or she can do so by pressing the first function button 25a. Accordingly, the graphical display 50 can display statistics for the portion of the therapy session completed, as shown in FIG. 13. Alternatively, if the practitioner wishes to continue the therapy session, he or she can press the second function button 25b.

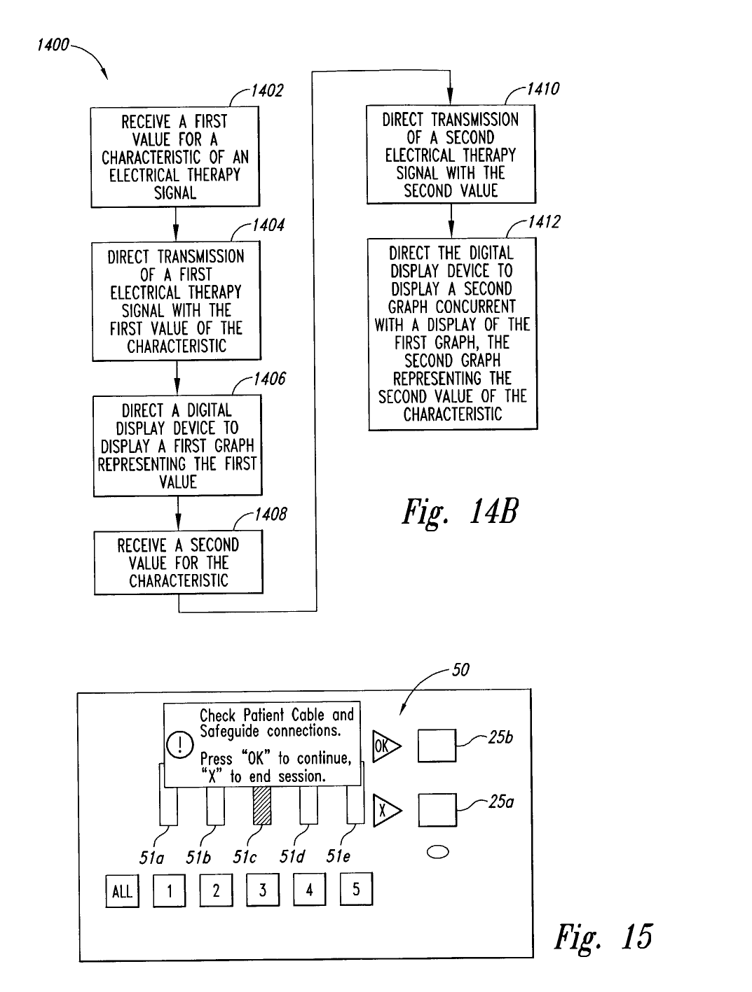

FIG. 14A illustrates the graphical display 50 as it can appear when the practitioner has resumed an initially paused therapy session by pressing the second function button 25b, as described above with reference to FIG. 12. As shown in FIG. 14A, each output bar 51 can include a previous current bar 56 (shown as bars 56a-e) having a reduced intensity and indicating the current level applied to that channel when the therapy session was paused. In one aspect of this embodiment, the control device can automatically bring the current level for each channel to zero as the session is resumed, regardless of the current level applied to the channels at the time the session was paused, and regardless of the position of the control knob 24 (FIG. 2). As the practitioner then increases the current level applied to the channels, the graphical display 50 can superimpose the present current bars 57 on the previous current bars 56. Accordingly, the practitioner can simultaneously view the previous current bar 56 and the current level presently applied to the recipient as the therapy session is resumed.

FIG. 14B is a flowchart illustrating a method 1400, such as a computer-implemented method, for displaying the information described above with reference to FIG. 14A in accordance with an embodiment of the invention. In one aspect of this embodiment, the method 1400 can include receiving a first value for a characteristic of an electrical therapy signal (step 1402). For example, step 1402 can include receiving a first level of electrical current to apply to a percutaneous electrical probe. In step 1404, the method can include transmitting a first electrical therapy signal with the first value of the characteristic. For example, step 1404 can include directing transmission of an electrical signal at the first current level to a probe inserted percutaneously in a recipient. In step 1406, the process can include directing a digital display device (such as the display screen 21 described above with reference to FIG. 2) to display a first graph representing the first value of the characteristic. For example, step 1406 can include directing the display screen 21 to display the previous current bars 56 (FIG. 14A).

In step 1408, the process can include receiving a second value for the characteristic. For example, step 1408 can include receiving a new current level to be applied to the recipient after a therapy session has been resumed. In step 1410, the process can include directing transmission of a second electrical therapy signal with the second value of the characteristic. In step 1412, the process can include directing the digital display device to display a second graph concurrent with the first graph and representing the second value of the characteristic. For example, step 1412 can include directing the display screen 21 (FIG. 2) to display the present current bars 57 (FIG. 14A) concurrently with the previous current bars 56. In a specific aspect of this embodiment, the present current bars 57 can be superimposed on the previous current bars 56. In either embodiment, the present current bars 57 and the previous current bars 56 can be displayed simultaneously to the practitioner so that the practitioner can compare the first and second values for the characteristic of the electrical therapy signal as the practitioner adjusts the second value. Instructions for performing any or all of the foregoing steps can be included in a computer-readable medium accessible to the control device 20 (FIG. 2). For example, the computer-readable medium can include a memory device 73 (such as a RAM device, ROM device, or removable media device) housed in or accessible by the control device 20.

FIG. 15 illustrates the graphical display 50 as it can appear when, during the course of a therapy session, one or more of the connections to the probe assemblies 32 (FIG. 1) become disrupted. The graphical display 50 can display a message indicating that the therapy session has been paused and, in a further aspect of this embodiment, can indicate which of the channels is affected, for example, by changing the color and/or intensity of output bar 51 for that channel (e.g., output bar 51c) relative to the remaining output bars (e.g., output bars 51a,b,d,e). In yet a further aspect of this embodiment, the control device 20 can automatically pause the delivery of electrical therapy signals to all channels when at least one connection has been disrupted. Once the connection to the affected channel has been reestablished (for example, by reconnecting a dislodged electrode), the practitioner can press the second function button 25b to resume the session, or press the first function button 25a to cancel the session.

One feature of the foregoing embodiments described above with reference to FIGS. 1-15, is that the control device 20 can include a single button (for example, the all-channel button 23) that the practitioner can activate to simultaneously control the current level applied to a plurality of output channels. An advantage of this feature is that it can save the practitioner time when initiating a new session or resuming a paused session. For example, if the practitioner knows that the recipient is more sensitive to electrical current delivered to one body location than another, the practitioner can, in one motion, increase the current level for all the channels to that threshold level, and then either further increase the current levels to all channels simultaneously, or individually tailor the current applied to each channel depending, for example, on the recipient's pain threshold.

In another aspect of this embodiment, the control device 20 can maintain the relative difference between currents applied to selected channels as the current levels to all channels are changed simultaneously. For example, if the current applied to the first channel is initially increased to a 50% level, and subsequently all the channels are increased together, then the first channel will reach a 100% level when the remaining channels reach the 50% level. If the current level applied to all the channels is further increased, the first channel will remain at 100% while the remaining channels increase up to 100%. If the current level is then decreased for all the channels, the first channel can remain at 100% until the remaining channels fall below 50%, at which point the current level applied to the first channel can fall as well.

Another feature of an embodiment of the control device 20 described above with reference to FIGS. 1-15 is that the graphical display 50 can include both a display of the previous current level applied to a particular channel, and the present current level. For example, the control device 20 can display both the current level applied to the channel(s) at the time the therapy session was paused, and the level applied as the therapy session is reinitiated. An advantage of this feature is that the practitioner can easily visualize the current level applied to the recipient at the time the session was paused, and can rapidly increase the current up to or near to that level when the therapy session is reinitiated. Accordingly, the practitioner can spend less time adjusting the current level applied to the recipient after the therapy session has been paused. An associated aspect of this feature is that the control device 20 can automatically apply a zero current level to each channel when the therapy session is restarted, to avoid suddenly applying a high current level to the recipient. The practitioner can then control the rate at which the current level is increased by manipulating the control knob 24 and visually monitoring the graphical display 50 and the recipient.

Still another feature of an embodiment of the control device 20 described above with reference to FIGS. 1-15 is that the control device 20 can automatically reset to zero the current level applied to each of the channels, not only when the therapy session is paused (as described above), but when a therapy session is completed. In either embodiment, the control device 20 can reset to zero the current level regardless of the position of the control knob 24. Accordingly, this aspect of an embodiment of the control device 20 differs from conventional control devices which may include potentiometers for current control. Such devices must be manually reset to zero before initiating a new therapy session, or the practitioner will risk applying a relatively high level of current to the recipient at the outset of a new therapy session or when a paused therapy session is resumed.

From the foregoing, it will be appreciated that specific embodiments of the invention have been described herein for purposes of illustration, but that various modifications may be made without deviating from the spirit and scope of the invention. The following examples provide further illustrations of embodiments of the invention.

I/We claim:

1. A computer-implemented method for displaying characteristics of an electrical therapy signal, comprising:
receiving a first value for a characteristic of an electrical therapy signal and directing transmission of the first electrical therapy signal with the first value of the characteristic;
directing a digital display device to display a first graph representing the first value of the characteristic;
receiving a second value for the characteristic and directing transmission of a second electrical therapy signal with the second value of the characteristic; and
directing the digital display device to display a second graph concurrent with a display of the first graph and representing the second value of the characteristic.

2. The computer-implemented method of claim 1 wherein receiving a first value for a characteristic of an electrical therapy signal includes receiving an electrical current value.

3. The computer-implemented method of claim 1 wherein directing a digital display device to display a first graph includes directing a digital display device to display a bar graph.

4. The computer-implemented method of claim 1 wherein directing a digital display device to display a first graph includes directing the digital display device to display a first graph having a first color, and wherein directing the digital display device to display a second graph includes directing the digital display device to display a second graph having a second color different than the first color.

5. The computer-implemented method of claim 1 wherein directing a digital display device to display a first graph includes directing the digital display device to display a first graph having a first intensity, and wherein directing the digital display device to display a second graph includes directing the digital display device to display a second graph having a second intensity different than the first intensity.

6. The computer-implemented method of claim 1 wherein directing a digital display device to display a first graph includes directing the digital display device to display a first bar graph, and wherein directing the digital display device to display a second graph includes directing the digital display device to display a second bar graph axially aligned with the first bar graph.

7. The computer-implemented method of claim 1, further comprising displaying an indication of a malfunction associated with a circuit carrying the first electrical therapy signal.

8. The computer-implemented method of claim 1, further comprising displaying an indication that the electrical therapy signal is selected for adjustment.

9. The computer-implemented method of claim 1 wherein directing the first electrical therapy signal includes directing the first electrical therapy signal to a percutaneous probe.

10. The computer-implemented method of claim 1, further comprising directing a digital display of a text message indicating the conclusion of a therapy session.

11. The computer-implemented method of claim 1 wherein receiving a first value includes receiving a first value for a portion of a therapy session, and wherein receiving a second value includes receiving a second value for a second portion of a therapy session subsequent to the first.

12. The computer-implemented method of claim 1 wherein receiving a first value includes receiving a first value for a therapy session and wherein receiving a second value includes receiving a second value for a second therapy session different than the first.

13. A computer-implemented method for displaying characteristics of an electrical therapy signal, comprising:
receiving a first value for a characteristic of an electrical therapy signal and directing transmission of a first electrical therapy signal with the first value of the characteristic;
directing a digital display device to display a first graph representing the first value of the characteristic, the first graph having a first color and a first intensity;
receiving a second value for the characteristic and directing transmission of a second electrical therapy signal with the second value of the characteristic; and
directing the digital display device to display a second graph superimposed on the first graph and representing the second value of the characteristic, the second graph having a second color and a second intensity, with the second color being different than the first color, or the second intensity being different than the first intensity, or both the second color being different than the first color and the second intensity being different than the first intensity.

14. The computer-implemented method of claim 13 wherein receiving a first value for a characteristic of an electrical therapy signal includes receiving an electrical current value.

15. The computer-implemented method of claim 13 wherein directing a digital display device to display a first graph includes directing the digital display device to display a first bar graph.

16. The computer-implemented method of claim 13 wherein directing a digital display device to display a first graph includes directing the digital display device to display a first bar graph, and wherein directing a digital display device to display a second graph includes directing a digital display device to display a second bar graph axially aligned with the first bar graph.

17. The computer-implemented method of claim 13, further comprising displaying an indication of a fault associated with a circuit carrying the first electrical therapy signal.

18. The computer-implemented method of claim 13, further comprising displaying an indication that the first electrical therapy signal is selected for adjustment.

19. The computer-implemented method of claim 13 wherein directing the first electrical therapy signal includes directing the first electrical therapy signal to a percutaneous probe.

20. The computer-implemented method of claim 13, further comprising directing a digital display of a text message indicating the conclusion of a therapy session.

21. A computer-implemented method for detecting characteristics of a percutaneous electrical therapy system, comprising:
detecting how many of a plurality of electrical therapy channels of a percutaneous electrical therapy system are coupled to conductive links for percutaneous electrical probes; and
deactivating at least one electrical therapy channel that is not coupled to a conductive link for a percutaneous electrical probe.

22. The computer-implemented method of claim 21 further comprising providing an indication to a user of the at least one deactivated electrical therapy channel.

23. The computer-implemented method of claim 21, further comprising deactivating an input button operatively coupled to the at least one electrical therapy channel.

24. A computer-implemented method for providing percutaneous electrical therapy, comprising:
detecting a characteristic of a signal link coupled to an electrical therapeutic signal emitter; and
based on the characteristic, selecting a characteristic of a signal transmitted on the signal link.

25. The method of claim 24 wherein detecting a characteristic of a signal link includes detecting information stored on a chip coupled to the signal link.

26. The method of claim 24 wherein selecting a characteristic of a signal includes selecting a current value of the signal.

27. The method of claim 24 wherein selecting a characteristic of a signal includes selecting a frequency with which the signal varies.

28. The method of claim 24 wherein selecting a characteristic of the signal includes selecting a duration of the signal.

29. The method of claim 24 wherein selecting a characteristic of the signal includes selecting a mode with which the signal is applied.

30. A method for providing percutaneous electrical therapy, comprising:
directing a first electrical therapy signal having a first value to a first site of a recipient;
directing a second electrical therapy signal having a second value to a second site of the recipient, the second value differing from the first value by a differential; and
changing the first and second values simultaneously while automatically maintaining the differential between the first and second values.

31. The method of claim 30 wherein changing the first and second values simultaneously includes decreasing the first and second values simultaneously.

32. The method of claim 30 wherein changing the first and second values simultaneously includes increasing the first and second values simultaneously.

33. A computer-readable medium having contents capable of performing a method for displaying characteristics of an electrical therapy signal, comprising:
receiving a first value for a characteristic of a first electrical therapy signal and directing transmission of the first electrical therapy signal with the first value of the characteristic;
directing a digital display device to display a first graph representing the first value of the characteristic;
receiving a second value for the characteristic and directing transmission of a second electrical therapy signal with the second value of the characteristic; and
directing the digital display device to display a second graph concurrent with a display of the first graph and representing the second value of the characteristic.

34. The computer-readable medium of claim 33 wherein receiving a first value for a characteristic of a first electrical therapy signal includes receiving an electrical current value.

35. The computer-readable medium of claim 33 wherein directing a digital display device to display a first graph includes directing a digital display device to display a bar graph.

36. The computer-readable medium of claim 33 wherein directing a digital display device to display a first graph includes directing a digital display device to display a first graph having a first color, and wherein directing a digital display device to display a second graph includes directing a digital display device to display a second graph having a second color different than the first color.

37. The computer-readable medium of claim 33 wherein directing a digital display device to display a first graph includes directing a digital display device to display a first graph having a first intensity, and wherein directing a digital display device to display a second graph includes directing a digital display device to display a second graph having a second intensity different than the first intensity.

38. The computer-readable medium of claim 33 wherein directing a digital display device to display a first graph includes directing a digital display device to display a first bar graph, and wherein directing a digital display device to display a second graph includes directing a digital display device to display a second bar graph axially aligned with the first bar graph.

39. The computer-readable medium of claim 33, further comprising displaying an indication of a malfunction associated with a circuit carrying the first electrical therapy signal.

40. The computer-readable medium of claim 33, further comprising displaying an indication that the electrical therapy signal is selected for adjustment.

41. The computer-readable medium of claim 33 wherein directing the first electrical therapy signal includes directing the first electrical therapy signal to a percutaneous probe.

42. The computer-readable medium of claim 33, further comprising directing a digital display of a text message indicating the conclusion of a therapy session.

43. An apparatus for controlling therapeutic electrical signals transmitted to a recipient, comprising:
a therapeutic signal emitter; and
a signal controller operatively coupled to the therapeutic signal emitter, the signal controller including a first input device coupled to a first channel to control a signal level directed to the first channel, the signal controller further including a second input device coupled to a second channel to control a signal level directed to the second channel independently of the signal level directed to the first channel, the signal controller still further including a common input device coupled to both the first and second channels to simultaneously control a single signal level directed to both the first and second channels.

44. The apparatus of claim 43 wherein the first input device includes a first pushbutton operatively coupled to a rotary knob and wherein the second input device includes a second pushbutton operatively coupled to the rotary knob.

45. The apparatus of claim 43 wherein the common input device includes a pushbutton operatively coupled to a rotary knob.

46. The apparatus of claim 43, further comprising a display device operatively coupled to the therapeutic signal emitter, the display device being configured to display characteristics of a therapeutic signal emitted by the therapeutic signal emitter.

47. The apparatus of claim 43, further comprising:
a support member;
first and second percutaneous probe actuators carried by the support member and configured to actuate percutaneous probes;
a first conductive link coupled between the first percutaneous actuator and the first channel; and
a second conductive link coupled between the second percutaneous actuator and the second channel.

48. An apparatus for providing percutaneous electrical therapy, comprising:
a signal emitter coupleable to a percutaneous probe and configured to emit an electrical signal; and
a signal input device operatively coupled to the signal emitter to control a current of the electrical signal emitted by the signal emitter, the signal input device being movable between a first position and a second position, the first position corresponding to a first non-zero electrical signal current and the second position corresponding to a second non-zero electrical current;
wherein the signal emitter is changeable from a powered state to an unpowered state, and wherein the signal emitter emits no electrical signal upon being placed in the powered state until the signal input device is moved, regardless of whether the signal input device is in the first position or the second position when the signal emitter is placed in the powered state.

49. The apparatus of claim 48 wherein the signal emitter is paused when in the unpowered state.

50. The apparatus of claim 48 wherein the signal input device includes a rotary knob.

51. The apparatus of claim 48, further comprising a display device operatively coupled to the signal emitter.
